(19) United States
(12) Patent Application Publication
(10) Pub. No.: US 2023/0293704 A1
(43) Pub. Date: Sep. 21, 2023

(54) Compositions and Methods for Tendon Regeneration

(71) Applicant: University of Rochester, Rochester, NY (US)

(72) Inventors: Danielle Benoit, Rochester, NY (US); Alayna E. Loiselle, Rochester, NY (US); Emmanuela Adjei-Sowah, Greece, NY (US)

(21) Appl. No.: 18/160,535

(22) Filed: Jan. 27, 2023

Related U.S. Application Data
(60) Provisional application No. 63/303,731, filed on Jan. 27, 2022.

(51) Int. Cl. A61K 47/60; A61K 45/06; A61K 47/64

(52) U.S. Cl. CPC A61K 47/60; A61K 45/06; A61K 47/64

(57) ABSTRACT

The present invention generally relates to novel compositions and methods for promoting regeneration of injured tendon. In some aspects, the composition comprises a nanoparticle that targets specifically to injured tendon to deliver a therapeutic agent that promotes tendon regeneration. In certain aspects, the methods comprise administering to a subject in need thereof a nanoparticle that targets specifically to injured tendon to deliver a therapeutic agent that promotes tendon regeneration.

### CROSS-REFERENCE TO RELATED APPLICATIONS

This application claims the benefit of priority from U.S. Provisional Application No. 63/303,731, filed Jan. 27, 2022, the which is hereby incorporated by reference in its entirety.

### STATEMENT REGARDING FEDERALLY SPONSORED RESEARCH OR DEVELOPMENT

This invention was made with government support under CBET1450987, awarded by the National Science Foundation, and 1R21AR081063, awarded by the National Institute of Arthritis and Musculoskeletal and Skin Diseases. The government has certain rights in the invention.

### REFERENCE TO A SEQUENCE LISTING IN XML FORMAT

The present application hereby incorporates by reference the entire contents of the XML file named “204606-0148-00US SequenceListing.xml” in XML format, which was created on Jan. 19, 2023, and is 5,319 bytes in size.

### BACKGROUND OF THE INVENTION

Tendon disorders are common and lead to significant disability, pain, healthcare cost, and lost productivity. A wide range of injury mechanisms exist leading to tendinopathy or tendon rupture. Tears can occur in healthy tendons that are acutely overloaded or lacerated. Tendinitis or tendinosis can occur in tendons exposed to overuse conditions (e.g., an elite swimmer's training regimen) or intrinsic tissue degeneration (e.g., age-related degeneration). The healing potential of a torn or pathologic tendon varies depending on anatomic location (e.g., Achilles vs. rotator cuff) and local environment (e.g., intrasynovial vs. extrasynovial). Although healing occurs to varying degrees, in general healing of repaired tendons follows the typical wound healing course, including an early inflammatory phase, followed by proliferative and remodeling phases. Numerous treatment approaches have been attempted to improve tendon healing, including growth factor- and cell-based therapies and rehabilitation protocols, with varying degrees of success (J Orthop Res. 2015 June; 33(6): 832-839).

Thus, there is a need in the art for improved compositions and methods for treating tendon injury. This invention satisfies this unmet need.

### SUMMARY OF THE INVENTION

In one embodiment, the present invention comprises a composition for controlled local delivery of a therapeutic agent to injured tendon, the composition comprising a targeting ligand tethered to a polymer and a therapeutic agent, wherein the therapeutic agent promotes tendon regeneration.

In one embodiment, the targeting ligand comprises a targeting ligand that specifically binds to a target associated with a site in need of tendon regeneration. In one embodiment, the targeting ligand is selected from the group consisting of: a nucleic acid, a peptide, an antibody, an antibody fragment, an inorganic molecule, an organic molecule, and any combination thereof.

In one embodiment, the targeting ligand comprises a peptide that specifically binds to tartrate-resistant acid phosphatase (TRAP). In one embodiment, the targeting ligand comprises TRAP Binding Peptide (TBP). In one embodiment, the targeting ligand comprises an amino acid sequence at least 95% identical to SEQ ID NO: 1. In one embodiment, the targeting ligand comprises the amino acid sequence of SEQ ID NO: 1.

In one embodiment, the therapeutic agent comprises one or more selected from the group consisting of: a nucleic acid, a peptide, an antibody, an antibody fragment, an inorganic molecule, an organic molecule, and any combination thereof. In one embodiment, the therapeutic agent comprises one or more selected from the group consisting of: a RAGE inhibitor, a RAGE receptor antagonist, an S100A4 inhibitor, a NFκB inhibitor, a NFκB-p65 inhibitor, a ROCK inhibitor, a TGF-β1 receptor antagonist, and an agent that reduces SMAD expression. In one embodiment, the therapeutic agent comprises one or more selected from the group consisting of: azeliragon, FPS-ZMI, niclosamide, pentamidine, Daxx, helenalin, parthenolide/micheliolide, Y27632, suramin, and halofuginone.

In one embodiment, the polymer is selected from the group consisting of poly(ethylene glycol) (PEG) methacrylate and poly(styrene-alt-maleic anhydride)-b-poly(styrene) (PSMA-b-PS).

In one embodiment, the present invention comprises a method of administering to a subject in need thereof a composition for use in promoting tendon regeneration, the method comprising administering to the subject a composition for controlled local delivery of a therapeutic agent to injured tendon, the composition comprising a targeting ligand tethered to a polymer and a therapeutic agent, wherein the therapeutic agent promotes tendon regeneration.

In one embodiment, the present invention comprises a method of promoting tendon regeneration at a site of tendon injury in a subject in need thereof, the method comprising administering to the subject a composition for controlled local delivery of a therapeutic agent to injured tendon, the composition comprising a targeting ligand tethered to a polymer and a therapeutic agent, wherein the therapeutic agent promotes tendon regeneration.

In one embodiment, the present invention comprises a method of treating tendon injury in a subject in need thereof, the method comprising administering to the subject a composition for controlled local delivery of a therapeutic agent to injured tendon, the composition comprising a targeting ligand tethered to a polymer and a therapeutic agent, wherein the therapeutic agent promotes tendon regeneration.

In some embodiments of the methods described, the subject has a disease or disorder selected from the group consisting of: tendonosis, tendonitis, tendinopathy, partial tendon rupture, and complete tendon rupture, age-related degeneration, and comorbidity-related degeneration.

In some embodiments of the methods described, the composition is administered during the late inflammatory and early proliferative stages of healing.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1D, depicts representative preparation and characterization of Trap binding protein-nanoparticles (TBP-NPs) via anhydride ring-opening. FIG. 1A depicts a schematic diagram depicting the ARO technique for conjugation of TBP to PSMA-b-Ps polymer and subsequent self-assembly into nanoparticles. FIG. 1B depicts representative nuclear magnetic resonance (NMR) traces demonstrating the presence of the Alloc protecting group, by its allylic protons, in Alloc-protected PSMA-b-Ps (top), and the subsequent absence of the allylic protons after conjugation to TBP (bottom). FIG. 1C depicts representative results of a binding assay demonstrating the lack of TRAP-targeting by SCP-NPs. FIG. 1D depicts representative results of a binding assay demonstrating that TBP-NPs effectively target and bind TRAP.

FIG. 2, comprising FIG. 2A through FIG. 2C, depicts a representative characterization of TBP-polymers when produced with varying feed ratios with normal TBP, TBP with an extra lysine (TBP-Lys⁺), without lysine (TBP-Lys⁻), or TBP with Alloc-protected lysine (TBP Alloc). FIG. 2A depicts representative characterization of the number-average molecular weight (Mₙ) of TBP-polymers produced through ARO conjugation. FIG. 2B depicts a representative characterization of the weight-average molecular weight (M_{w}) of TBP-polymers produced through ARO conjugation. FIG. 2C depicts a representative dispersity characterization (Ð) of TBP-polymers produced through ARO conjugation. n=3; &, p<0.05 between TBP and TBP-Lys⁻; $, p<0.05 between TBP and TBP Alloc; @, p<0.05 between TBP and TBP-Lys⁺; #, p<0.05 between TBP-Lys⁺ and TBP-Lys⁻; %, p<0.05 between TBP-Lys⁺ and TBP Alloc.

FIG. 3, comprising FIG. 3A through FIG. 3C, depicts representative results demonstrating that a spatial transcriptomic analysis of tendon healing identifies an inflammatory/macrophage cluster at the tendon repair site. FIG. 3A depicts a representative UMAP analysis of unsupervised clustering of spatial transcriptomics data from uninjured tendons and tendons at 14- and 28-days post-repair, identifying 5 distinct molecular clusters. FIG. 3B depicts Cluster 4, which is defined as an inflammatory, macrophage enriched cluster, further defined, in part, by high expression of Acp5. FIG. 3C depicts representative mapping of Acp5, demonstrating high expression and specific localization in the tendon stubs (black) and bridging tissue (green).

FIG. 4, comprising FIG. 4A through FIG. 4C, depicts representative results demonstrating that the cluster annotated as Macrophage 1 express Acp5 after tendon injury. FIG. 4A depicts a representative UMAP analysis of unsupervised clustering of spatial transcriptomics data from uninjured tendons and tendons at 7-, 14, and 28-days post-repair identifying different molecular clusters. FIG. 4B depicts temporal induction of Acp5 in the macrophage 1 cluster. FIG. 4C depicts representative results demonstrating an increase in Acp-5 expression in macrophage 1 after injury, in stark contrast to macrophage 2 cluster.

FIG. 5, comprising FIG. 5A through FIG. 5C, depicts representative measurements of regenerative, tenogenic healing in S100a4^{GFP/+} mice 14 days post-surgery. FIG. 5A depicts a representative decrease in scar tissue volume in S100a4^{GFP/+} mice relative to wildtype. FIG. 5B depicts a representative increase in the range of motion in S100a4^{GFP/+} mice relative to wildtype mice. FIG. 5C depicts a representative increase in weight-bearing capabilities in tendons of S100a4^{GFP/+} mice relative to wildtype mice.

FIG. 6 depicts representative results demonstrating TRAP+ cells at the tendon repair site. High levels of TRAP activity (red) are observed in the healing tendon. At day 7 a cluster of TRAP cells is observed in the bridging tissue (outlined in red), with an additional TRAP+ population in tendon stub (outlined in black). By day 14 the TRAP+ population has expanded with diffuse localization throughout both the tendon stub and bridging scar tissue. Sections are counterstained with methyl green.

FIG. 7, comprising FIG. 7A and FIG. 7B, depicts representative results demonstrating, via in vivo imaging, enhanced targeting of the healing tendon with IR780-labeled TRAP Binding Peptide nanoparticles (TBP-NPs) compared to IR780-labeled scrambled peptide nanoparticles (SCP-NPs). Nanoparticles were administered via retroorbital injection three days following tendon transection and repair. FIG. 7A depicts representative IVIS images of IR780-NP localization 1-14 days post-injection. Robust recruitment of TBP-NPs was observed at the tendon repair site while minimal localization was observed in SCP-NP treated animals. FIG. 7B depicts representative results of radiant efficiency (normalized to saline treated controls) from 1-14 days post-NP injection. N=5; *, p<0.05 by two-way ANOVA.

FIG. 8, comprising FIG. 8A and FIG. 8B, depicts representative results demonstrating enhanced targeting of the healing tendon with IR780-labeled TBP-NPs compared to IR780-labeled SCP-NPs. Nanoparticles were administered via retroorbital injection seven days following tendon transection and repair. FIG. 8A depicts representative IVIS images of IR780-NP localization 1-14 days post-injection. Robust recruitment of TBP-NPs was observed at the tendon repair site while minimal localization was observed in SCP-NP treated animals. FIG. 8B depicts representative results of radiant efficiency (normalized to saline treated controls) from 1-14 days post-NP injection. N=5; *, p<0.05 by two-way ANOVA.

FIG. 9, comprising FIG. 9A and FIG. 9B, depicts representative results demonstrating enhanced targeting of the healing tendon with IR780-labeled TBP-NPs compared to IR780-labeled SCP-NPs. Nanoparticles were administered via retroorbital injection 14 days following tendon transection and repair. FIG. 9A depicts representative IVIS images of IR780-NP localization 1-14 days post-injection. Robust recruitment of TBP-NPs was observed at the tendon repair site while minimal localization was observed in SCP-NP treated animals. FIG. 9B depicts representative results of radiant efficiency (normalized to saline treated controls) from 1-14 days post-NP injection. N=5; *, p<0.05 by two-way ANOVA.

FIG. 10 depicts representative results of radiant efficiency (normalized to saline treated controls) from 1-14 days post-NP injection for mice treated 3, 7, or 14 days after tendon transection and repair.

FIG. 11, comprising FIG. 11A and FIG. 11B, depicts representative results demonstrating targeting of the healing tendon with IR780-label TBP-NPs compared to IR780-labeled SCP-NPs when dosed at 5 mg/kg. FIG. 11A depicts representative IVIS images of IR780-NP localization 1-14 days post-injection. FIG. 11B depicts representative results of radiant efficiency (normalized to saline-treated controls) from 1-14 days post-NP injection.

FIG. 12, comprising FIG. 12A and FIG. 12B, depicts representative results demonstrating targeting of the healing tendon with IR780-label TBP-NPs compared to IR780-labeled SCP-NPs when dosed at 25 mg/kg. FIG. 12A depicts representative IVIS images of IR780-NP localization 1-14 days post-injection. FIG. 12B depicts representative results of radiant efficiency (normalized to saline-treated controls) from 1-14 days post-NP injection.

FIG. 13, comprising FIG. 13A and FIG. 13B, depicts representative results demonstrating enhanced targeting of the healing tendon with IR780-label TBP-NPs compared to IR780-labeled SCP-NPs when dosed at 50 mg/kg. FIG. 13A depicts representative IVIS images of IR780-NP localization 1-14 days post-injection. FIG. 13B depicts representative results of radiant efficiency (normalized to-saline treated controls) from 1-14 days post-NP injection.

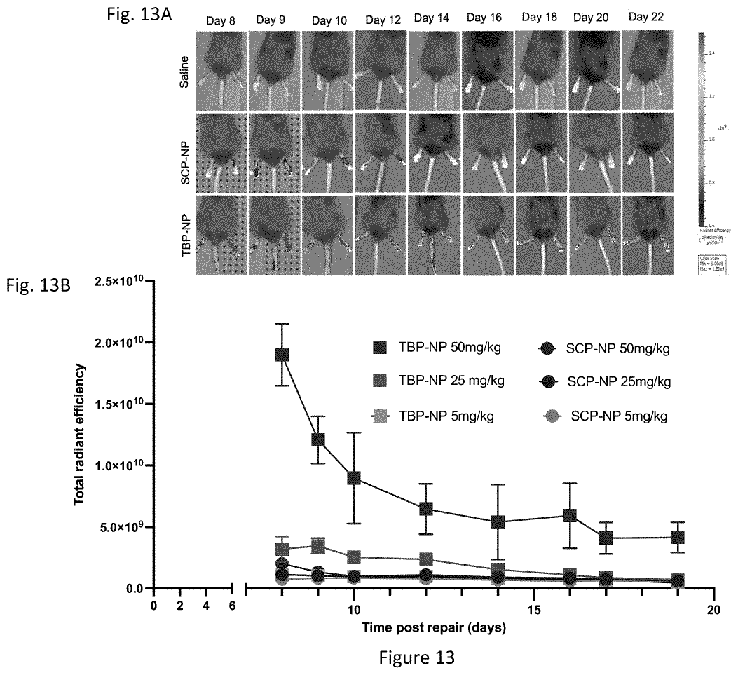

FIG. 14 depicts a schematic representation of the process of polymer synthesis, nanoparticle formation, peptide-polymer conjugation, and loading of a tendon regeneration drug.

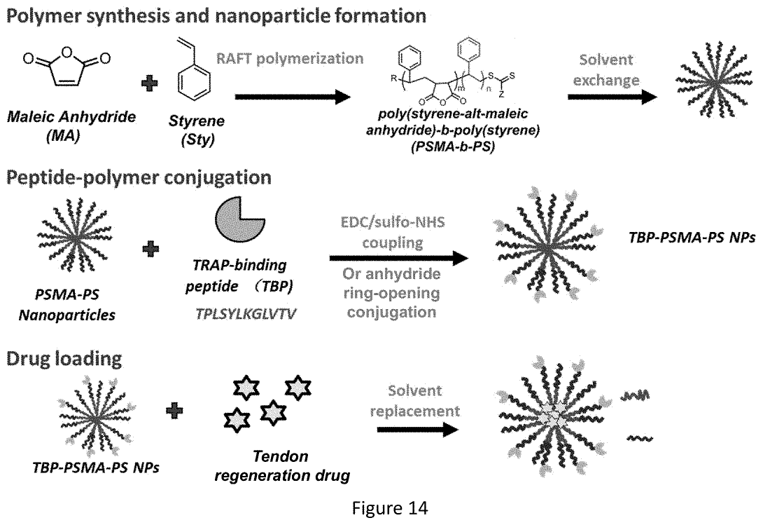

### DETAILED DESCRIPTION

The present invention generally relates to compositions and methods for promoting or enhancing regeneration of damaged tendon. The present invention is based, in part, upon the discovery that Acp5, the gene encoding tartrate resistant acid phosphatase (TRAP), is highly expressed in healing tendons, and that TRAP binding peptide (TBP) nanoparticles (TBP-NPs) can accumulate at these sites of injury.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used here, “biocompatible” refers to any material that, when implanted in a mammal, does not provoke an adverse response in the mammal. When introduced into an individual, a biocompatible material is not toxic or injurious to that individual, nor does it induce immunological rejection of the material in the mammal. “Biocompatible” also refers to a property of a composition characterized by its degradation products or its in vivo degradation products being not, or at least is minimally and/or reparably, injurious to living tissue; and/or not, or at least minimally and controllably, causing an immunological reaction in living tissue.

A “disease” is a state of health of an animal wherein the animal cannot maintain homeostasis, and wherein if the disease is not ameliorated, the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Unless otherwise specified, a “nucleotide sequence (or nucleic acid molecule) encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of a composition includes, e.g., subcutaneous (s.c.), intravenous (i.v.), intramuscular (i.m.), or intrasternal injection, or infusion techniques. “Enteral” administration of a composition generally refers to delivery involving any part of gastrointestinal tract including oral delivery and rectal delivery. Parenteral and enteral administration have systemic effects.

The terms “polynucleotide”, “nucleic acid” and “nucleic acid molecule” as used herein interchangeably, are defined as a chain of nucleotides. Furthermore, nucleic acids are polymers of nucleotides. One skilled in the art has the general knowledge that nucleic acids are polynucleotides, which can be hydrolyzed into the monomeric “nucleotides.”

The monomeric nucleotides can be hydrolyzed into nucleosides. As used herein polynucleotides include, but are not limited to, all nucleic acid sequences that are obtained by any means available in the art, including, without limitation, recombinant means, i.e., the cloning of nucleic acid sequences from a recombinant library or a cell genome, using ordinary cloning technology and PCR™, and the like, and by synthetic means.

In some instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “polymer” refers to a molecule composed of repeating structural units typically connected by covalent chemical bonds. The term “polymer” is also meant to include the terms copolymer and oligomers.

As used herein, the term “polymerization” refers to at least one reaction that consumes at least one functional group in a monomeric molecule (or monomer), oligomeric molecule (or oligomer) or polymeric molecule (or polymer), to create at least one chemical linkage between at least two distinct molecules (e.g., intermolecular bond), at least one chemical linkage within the same molecule (e.g., intramolecular bond), or any combination thereof. A polymerization reaction may consume between about 0% and about 100% of the at least one functional group available in the system. In one embodiment, polymerization of at least one functional group results in about 100% consumption of the at least one functional group. In another embodiment, polymerization of at least one functional group results in less than about 100% consumption of the at least one functional group.

The term “promoter” as used herein is defined as a DNA sequence recognized by the synthetic machinery of the cell, or introduced synthetic machinery, required to initiate the specific transcription of a polynucleotide sequence.

As used herein, the term “patient,” “subject,” “individual,” and the like are used interchangeably herein, and refer to any animal, or cells thereof, whether in vitro or in situ, amenable to the methods described herein. In certain non-limiting embodiments, the patient, subject or individual is a mammal, non-limiting examples of which include a primate, dog, cat, goat, horse, pig, mouse, rat, rabbit, and the like, that is in need of bone formation or bone treatment. In some embodiments of the present invention, the subject is a human being. In such embodiments, the subject is often referred to as an “individual” or a “patient.” The terms “individual” and “patient” do not denote any particular age.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

A “vector” is a composition of matter that comprises an isolated nucleic acid and that can be used to deliver the isolated nucleic acid to the interior of a cell. Numerous vectors are known in the art, including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention generally relates to compositions and methods for promoting tendon regeneration in a subject in need thereof. In one embodiment, the present invention relates to a composition for controlled local delivery of a therapeutic agent to injured tendon. In one embodiment, the composition comprises a targeting ligand. In one embodiment, the composition comprises a therapeutic agent. In one embodiment, said composition comprises a nanoparticle. In one embodiment, the nanoparticle comprises a polymer. In one embodiment, the nanoparticle comprises a tether conjugating the polymer to the targeting ligand.

In some embodiments, the subject in need thereof has a disease or disorder associated with tendon injury. Representative embodiments include, but are not limited to, tendonosis, tendonitis, tendinopathy, partial tendon rupture, and complete tendon rupture, age-related tendon degeneration, and comorbidity-related tendon degeneration (e.g., diabetes).

### Nanoparticles

In some embodiments, the composition for controlled local delivery of a therapeutic agent to injured tendon comprises a nanoparticle. In certain embodiments, nanoparticles provided herein selectively uptakes small hydrophobic molecules, such as hydrophobic small molecule compounds (e.g., hydrophobic small molecule drugs) into the hydrophobic core of the particle. In certain embodiments, the nanoparticle provided herein comprises a therapeutic agent conjugated by way of linkers and/or tethers to one or more components of the nanoparticle.

In some embodiments, the nanoparticles provided herein retain activity (e.g., the activity nanoparticle to deliver a therapeutic agent) in mammalian tissue for at least 2 hours, at least 4 hours, at least 6 hours, at least 8 hours, at least 12 hours, or at least 24 hours.

In certain embodiments, the nanoparticle has a size of approximately 10 nm to about 200 nm, about 10 nm to about 100 nm, or about 30-80 nm. Particle size can be determined in any manner, including, but not limited to, by gel permeation chromatography (GPC), dynamic light scattering (DLS), electron microscopy techniques (e.g., TEM), and other methods.

In one embodiment, the nanoparticle comprises a hydrophobic core region and a hydrophilic shell region. For example, in one embodiment, the nanoparticle comprises copolymers, comprising at least one hydrophobic region and at least one hydrophilic region. In certain embodiments, the therapeutic agent described herein is covalently or noncovalently attached to the nanoparticle.

The nanoparticle of the invention may be formed by any suitable method known in the art or hereafter developed. In certain embodiments, polymers comprised in the nanoparticle of the invention are synthesized using reversible addition-fragmentation chain transfer (RAFT). This technique uses a chain transfer agent (CTA) capable of maintaining the radical state of the propagating species or reinitiating polymerization and yields polymers with a low polydispersity index (PDI), indicative of uniform polymers. RAFT is a chain polymerization that introduces a CTA that modulates the rate of reaction thus forming polymers with well-controlled molecular weights and polydispersities, polymer chain ends with different functionalities, and a multitude of possible architectures. These characteristics are inherently important for reproducible therapeutic manufacturing, while the large variety in possible architectures enables design-on-demand methods to address the requirements of the delivery system. For example, dendrimers or brush architectures, as well as the end-functional nature of these polymers, impart the ability to mix-and-match drugs, targeting, or other functional moieties. This provides easy incorporation of both multivalent targeting and drug delivery chemistries into polymer architectures for tissue-specific delivery. In addition, RAFT polymers improve stability of therapeutic molecules, reduce immunogenicity, enhance solubility, and increase blood circulation times to achieve high doses of therapeutic at the right time, at the right place, and at the right concentrations. However, the polymers of the invention are not limited to polymers synthesized by RAFT. Other suitable methods include, but are not limited to emulsion polymerizations, atom-transfer radical polymerization (ATRP), traditional chain polymerization, and step polymerization. Representative methods of polymerization, and copolymerization are well known in the art such as those discussed in De Souza Gomes (2012, Polymerization, InTech).

In some embodiments, the nanoparticles of the present invention comprise a targeting ligand.

In specific embodiments, the compositions provided herein are biocompatible, as defined elsewhere herein. With regard to salts, it is presently preferred that both the cationic and the anionic species be biocompatible or “physiologically acceptable,” which is interchangeable with biocompatible herein. In some instances, the polymer bioconjugates and polymers used herein (e.g., copolymers) exhibit low toxicity compared to cationic lipids.

### Polymers

In some embodiments, the composition of the invention comprises one or more polymer. In some embodiments, the nanoparticle of the invention comprises one or more polymer. In certain embodiments, the nanoparticle of the invention comprises a polymer of 1, 2, 5, 10, or more different types of monomers. The polymer can be manufactured to have a variety of different polymer architectures that allow the particle to have improved stability. In one embodiment, the particle comprises a homopolymer comprising the therapeutic agent. In another embodiment, the particle comprises a copolymer comprising the therapeutic agent. Copolymers can have a variety of different architectures that, in certain embodiments, may be preferred to allow for 1) targeting of the particle, 2) controlled release of the therapeutic, and/or 3) stability of the particle. Representative architectures of copolymers include, but are not limited to, diblock copolymers, random copolymers, statistical copolymers, gradient copolymers, graft copolymers, and dendrimer copolymers. Representative polymers that may be used in the copolymer include, but are not limited to, PEG, PLGA, PEG methacrylate, polystyrene, polymethacrylate, polyacrylamide, PSMA-b-PS methacrylate, and the like.

In certain embodiments, the polymers described herein are synthesized using reversible addition-fragmentation chain transfer (RAFT) polymerization. RAFT polymerization is a controlled living polymerization strategy for developing polymers with well-controlled molecular weights and polydispersities, polymer chain ends with different end functionalities, and a variety of architectures. In certain embodiments, these characteristics are beneficial for the development of effective and easy to manufacture polymer-based therapeutics.

In one embodiment, RAFT polymerization is used to covalently conjugate maleic anhydride (MA) and styrene (Sty) to form the polymer poly(styrene-alt-maleic anhydride)-b-poly(styrene) (PSMA-b-PS). In some embodiments, 1-Ethyl-3-[3-dimethylaminopropyl]carbodiimide (EDC) and N-hydroxysulfosuccinimide (sulfo-NHS) are used to couple the polymer to a targeting peptide. In some embodiments, solvent exchange is used to convert the individual polymers to a nanoparticle. In some embodiments, solvent replacement is used to load a hydrophobic therapeutic agent into the hydrophobic core of the nanoparticle.

In various embodiments, copolymers utilized in the nanoparticles described herein have or are selected to have an influence on a certain aspect or functionality of the nanoparticles provided herein, including but not limited to: (1) the biophysical properties of the nanoparticle such as, by way of non-limiting example, solubility, aqueous solubility, stability, stability in an aqueous medium, hydrophilicity, lipophilicity, hydrophobicity, or the like; (2) the facilitation of the formulation of the nanoparticle into an administrable form, or other purposes; (3) the ability of the nanoparticle to target a specific or selected type of cell or biostructure (e.g., by carrying a targeting moiety); and/or (4) the ability to increase biocompatibility of the nanoparticle. In some embodiments, a nanoparticle provided herein is characterized by one or more of the following: (1) the nanoparticle is formed by spontaneous self-association of copolymers to form organized assemblies (e.g., nanoparticles) upon dilution from a water-miscible solvent (such as but not limited to ethanol) to aqueous solvents (for example phosphate-buffered saline, pH 7.4); (2) the nanoparticle is stable to dilution (e.g., down to a polymer concentration of 100 μg/ml, 50 μg/ml, 10 μg/ml, 5 μg/ml or 1 μg/ml, which constitutes the critical stability concentration or the critical micelle concentration (CMC) or critical nanoparticle concentration (CNC)); (3) the nanoparticle is stable to high ionic strength of the surrounding media (e.g. 0.5M NaCl); and/or (4) the nanoparticle has an increasing instability as the concentration of organic solvent increases, such organic solvents including, but not limited to dimethylformamide (DMF), dimethyl sulfoxide (DMSO), and dioxane. In some embodiments, a nanoparticle provided herein is characterized by having at least two of the aforementioned properties. In some embodiments, a nanoparticle provided herein is characterized by having at least three of the aforementioned properties. In some embodiments, a nanoparticle provided herein is characterized by having all of the aforementioned properties.

In some embodiments, the polymer is selected from the group including, but not limited to, poly(ethylene glycol) (PEG) methacrylate and poly(styrene-alt-maleic anhydride)-b-poly(styrene) (PSMA-b-PS).

### Targeting Ligands

In some embodiments, the composition of the present invention comprises a targeting ligand. In some embodiments, the nanoparticle of the present invention comprises a targeting ligand. A skilled artisan will recognize that any targeting ligand suitable for directing one or more component of the composition to a site in need of tendon repair or regeneration can be used in the methods of the present invention. Representative targeting ligands include, but are not limited to, nucleic acids, peptides, antibodies, antibody fragments, inorganic molecules, organic molecules, and any combinations thereof.

In some embodiments, the composition comprises a targeting ligand that specifically binds to a target associated with a site in need of tendon repair or regeneration. In some embodiments, the target associated with a site in need of tendon repair regeneration comprises tartrate-resistant acid phosphatase (TRAP). As demonstrated herein, TRAP is upregulated in tendons following injury and during regeneration, and thus is associated with a site that is in need of tendon regeneration.

In some embodiments, the targeting ligand comprises an antibody or antibody fragment that specifically binds to TRAP. Antibodies and fragment thereof can be produced by a variety of methods described elsewhere herein. In one embodiment, the targeting domain may consist of an immunoglobulin (Ig) heavy chain which may in turn be covalently associated with an Ig light chain by virtue of the presence of CH1 and hinge regions or may become covalently associated with other Ig heavy/light chain complexes by virtue of the presence of hinge, CH2 and CH3 domains. In the latter case, the heavy/light chain complex that becomes joined to the chimeric construct may constitute an antibody with a specificity distinct from the antibody specificity of the chimeric construct. Depending on the function of the antibody, the desired structure and the signal transduction, the entire chain may be used, or a truncated chain may be used, where all or a part of the CH1, CH2, or CH3 domains may be removed, or all or part of the hinge region may be removed.

In some embodiments, the targeting ligand comprises a targeting peptide that specifically binds to TRAP. Peptide and peptide fragments can be manufactured using biological or synthetic techniques, as described elsewhere herein. Further, the targeting ligand encompasses chimeric peptides, peptidomimetics, and peptide variants, as discussed elsewhere herein.

In one embodiment, the targeting peptide comprises TRAP Binding Peptide (TBP), or a fragment or variant thereof. TBP is a peptide known to home to TRAP with sub-nanomolar affinity.

In one embodiment, the targeting peptide comprises an amino acid sequence at least 80%, at least 85%, at least 90%, at least 95%, at least 97%, at least 98%, or at least 99% identical to SEQ ID NO: 1. In one embodiment, the targeting peptide comprises the amino acid sequence TPLSYLKGLVTVG (SEQ ID NO: 1).

In one embodiment, the targeting peptide comprises an amino acid sequence at least 80%, at least 85%, at least 90%, at least 95%, at least 97%, at least 98%, or at least 99% identical to SEQ ID NO: 2. In one embodiment, the peptide comprises the amino acid sequence TPLSYLKGLVTV (SEQ ID NO: 2).

In one embodiment, the targeting peptide comprises a methacrylamide group. Functionalization with a polymerizable methacrylamide group provides one non-limiting example of methods that allow for incorporating a peptide into a polymer.

In one embodiment, the targeting peptide of the nanoparticle, as described herein is attached to either end of a polymer or to a side chain or a pendant group of a monomeric unit, or to the end of a backbone polymer, or incorporated into a polymer. In some instances, the targeting peptide is covalently coupled to the polymer of the nanoparticle at the opposite end from the therapeutic agent or the hydrophobic core of the nanoparticle where a hydrophobic therapeutic agent resides.

Attachment of a targeting peptide to the polymer can be achieved in any suitable manner, e.g., by any one of a number of conjugation chemistry approaches including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In specific embodiments, “click” chemistry is used to attach the targeting ligand to the polymers of the polymer bioconjugates provided herein (for example of “click” reactions, see Wu, P.; Fokin, V. V. Catalytic Azide-Alkyne Cycloaddition: Reactivity and Applications. Aldrichim. Acta 2007, 40, 7-17). A large variety of conjugation chemistries are optionally utilized (see, for example, Bioconjugation, Aslam and Dent, Eds, Macmillan, 1998 and chapters therein). In some embodiments, targeting peptide are attached to a monomer and the resulting compound is then used in the polymerization synthesis of a polymer (e.g., copolymer).

### Therapeutic Agents

In some embodiments, the composition of the invention comprises a therapeutic agent. In one embodiment, composition of the present invention comprises a nanoparticle and a therapeutic agent. In some embodiments, the nanoparticle encapsulates the therapeutic agent. In some embodiments, the therapeutic agent is covalently conjugated to the nanoparticle. In other embodiments, the therapeutic agent is not covalently conjugated to the nanoparticle. The therapeutic agent can be any known therapeutic that promotes tendon regeneration, including but not limited to, a nucleic acid, protein, peptide, small molecule, aptamer, antagonist, peptidomimetic, or combination thereof. For example, in certain embodiments, the therapeutic agent may enhance the expression or activity of a biomolecule known to play a role in tendon regeneration. By way of further example, in certain embodiments, the therapeutic agent may decrease the expression or activity of a biomolecule known to inhibit tendon regeneration.

Nucleic Acids

In some embodiments, the therapeutic agent comprises a nucleic acid molecule. In one embodiment, the nucleic acid molecule encodes a therapeutic protein. In one embodiment, the therapeutic protein promotes tendon regeneration. In one embodiment, the therapeutic protein decreases the activity or expression of a biomolecule that inhibits tendon regeneration.

A nucleic acid molecule encoding a therapeutic protein of the invention (e.g., that promotes tendon regeneration or that decreases the activity or expression of a biomolecule that inhibits tendon regeneration) can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

A nucleic acid molecule may comprise any type of nucleic acid, including, but not limited to DNA and RNA. For example, in one embodiment, the composition comprises an isolated DNA molecule, including for example, an isolated cDNA molecule, encoding a therapeutic protein of the invention. In one embodiment, the composition comprises an isolated RNA molecule encoding a therapeutic protein of the invention, or a functional fragment thereof.

In one embodiment, the composition comprises nucleoside-modified RNA. Nucleoside-modified RNA has particular advantages over non-modified RNA, including for example, increased stability, low or absent innate immunogenicity, and enhanced translation. Nucleoside-modified mRNA useful in the present invention is further described in U.S. Pat. Nos. 8,278,036, 8,691,966, and 8,835,108, each of which is incorporated by reference herein in its entirety.

In one embodiment, the present invention comprises a nucleic acid for exogenous introduction into one or more cell. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

The present invention also includes a vector in which the nucleic acid molecule of the present invention is inserted. The art is replete with suitable vectors that are useful in the present invention.

In brief summary, the expression of natural or synthetic nucleic acids encoding a therapeutic protein of the invention is typically achieved by operably linking a nucleic acid encoding the therapeutic protein of the invention or portions thereof to a promoter, and incorporating the construct into an expression vector. The vectors to be used are suitable for replication and/or integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid molecule of the invention can be cloned into a number of types of vectors. For example, the nucleic acid can be cloned into a vector including, but not limited to a plasmid, a phagemid, a phage derivative, an animal virus, and a cosmid. Additional vectors include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In one embodiment, the nucleic acid molecule directly decreases the expression or activity of a biomolecule that inhibits tendon regeneration. Representative nucleic acid molecules suitable for this purpose include, but are not limited to, siRNA, microRNA, shRNA, antisense nucleic acids, ribozymes, killer-tRNAs, guide RNAs (part of the CRISPR/CAS system), long non-coding RNA, anti-miRNA oligonucleotides, and plasmid DNA.

RNA interference (RNAi) is normally triggered by double stranded RNA (dsRNA) or endogenous microRNA precursors (pri-miRNAs/pre-miRNAs). Since its discovery, RNAi has emerged as a powerful genetic tool for suppressing gene expression in mammalian cells. Stable gene knockdown can be achieved by expression of synthetic short hairpin RNAs (shRNAs). In one embodiment, the therapeutic agent comprises a nucleic acid molecule. The nucleic acid molecule may be DNA, RNA, cDNA, microRNA, siRNA, shRNA, or the like.

In some embodiments, the therapeutic agent comprises siRNA polynucleotide. An siRNA polynucleotide is an RNA nucleic acid molecule that interferes with RNA activity that is generally considered to occur via a post-transcriptional gene silencing mechanism. An siRNA polynucleotide preferably comprises a double-stranded RNA (dsRNA) but is not intended to be so limited and may comprise a single-stranded RNA (see, e.g., Martinez et al., 2002 Cell 110:563-74). The siRNA polynucleotide included in the invention may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly, it will be appreciated that certain representative sequences disclosed herein as DNA sequences capable of directing the transcription of the siRNA polynucleotides are also intended to describe the corresponding RNA sequences and their complements, given the well-established principles of complementary nucleotide base-pairing.

The siRNA polynucleotide can be cloned into a number of types of vectors as described elsewhere herein. For expression of the siRNA or antisense polynucleotide, at least one module in each promoter functions to position the start site for RNA synthesis.

To assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

In some embodiments, the therapeutic agent comprises an antisense nucleic acid molecule. Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Antisense molecules of the invention may further be made synthetically and then provided to the cell. Antisense oligomers of between about 10 to about 30, and more preferably about 15 nucleotides, are preferred since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In some embodiments, the therapeutic agent comprising a nucleic acid molecule comprises an antisense nucleic acid sequence which is expressed by a plasmid vector. The antisense expressing vector is used to transfect a mammalian cell or the mammal itself, thereby causing reduced endogenous expression of a desired regulator in the cell. The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

In some embodiments, the therapeutic agent comprises a ribozyme. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence specific.

There are two basic types of ribozymes, namely, tetrahymena-type (Hasselhoff, 1988, Nature 334:585) and hammerhead-type. Tetrahymena-type ribozymes recognize sequences that are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes may be preferable to tetrahymena-type ribozymes for inactivating specific mRNA species, and 18-base recognition sequences may be preferable to shorter recognition sequences which may occur randomly within various unrelated mRNA molecules.

In some embodiments of the invention, a miRNA or a synthetic miRNA is used as a therapeutic agent to regulate gene expression. The miRNA may contain one or more design elements. These design elements include but are not limited to: i) a replacement group for the phosphate or hydroxyl of the nucleotide at the 5′ terminus of the complementary region; ii) one or more sugar modifications in the first or last 1 to 6 residues of the complementary region; or, iii) noncomplementarity between one or more nucleotides in the last 1 to 5 residues at the 3′ end of the complementary region and the corresponding nucleotides of the miRNA region.

Any nucleic acid molecule described herein may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

Therapeutic Proteins

In some embodiments, the therapeutic agent is a therapeutic protein. In one embodiment, the therapeutic protein promotes tendon regeneration. In one embodiment, the therapeutic protein decreases the activity or expression of a biomolecule that inhibits tendon regeneration.

A therapeutic protein of the invention may be synthesized by conventional techniques. For example, peptides may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a therapeutic protein of the invention conjugated with other molecules may be prepared by fusing, through recombinant techniques, the N-terminal or C-terminal of the therapeutic protein, and the sequence of a selected protein or selectable marker with a desired biological function. The resultant fusion proteins contain the fusion protein fused to the selected protein or marker protein as described herein. Examples of proteins that may be used to prepare fusion proteins include immunoglobulins, glutathione-S-transferase (GST), hemagglutinin (HA), and truncated myc.

Therapeutic proteins of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The therapeutic proteins of the invention may be converted into pharmaceutical salts by reacting with inorganic acids, including but not limited to, hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In some embodiments, the therapeutic protein is an antibody. The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin.

As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, NY). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX. Monoclonal antibodies directed against full length or peptide fragment of a protein or peptide may be prepared using any well-known monoclonal antibody preparation procedures, such as those described, for example, in Harlow et al. (1988, In: Antibodies, A Laboratory Manual, Cold Spring Harbor, NY) and in Tuszynski et al. (1988, Blood, 72:109-115).

Prior to its use, a therapeutic protein is purified to remove contaminants. In this regard, it will be appreciated that the therapeutic protein will be purified so as to meet the standards set out by the appropriate regulatory agencies. Any one of a number of conventional purification procedures may be used to attain the required level of purity, including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Antibodies and proteins may be modified using ordinary molecular biological techniques to improve their resistance to proteolytic degradation, or to optimize solubility properties, or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

Small Molecules

In one embodiment, the therapeutic agent of the present invention comprises one or more small molecule. In one embodiment, the therapeutic agent includes, but is not limited to, a Receptor for Advanced Glycation Endproducts (RAGE) inhibitor, a RAGE receptor antagonist, an S100 calcium-binding protein A4 (S100A4) inhibitor, a Nuclear Factor Kappa B (NFκB) inhibitor, a NFκB-p65 inhibitor, a Rho-associated protein kinase (ROCK) inhibitor, a transforming growth factor beta-1 (TGF-β1) receptor antagonist, and an agent that reduces SMAD expression.

In one embodiment, the therapeutic agent includes, but is not limited to, azeliragon, FPS-ZMI, niclosamide, pentamidine, Daxx, helenalin, parthenolide/micheliolide, Y27632, suramin, and halofuginone.

When the therapeutic agent of the invention is a small molecule, such a small molecule may be obtained using standard methods known to the skilled artisan. Such methods include chemical organic synthesis or biological means. Biological means include purification from a biological source, recombinant synthesis, and in vitro translation systems, using methods well known in the art. In some embodiments, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule compounds described herein may be present as salts even if salts are not depicted, and it is understood that the invention embraces all salts and solvates of the small molecules described herein, as well as the non-salt and non-solvate form of the small molecules, as is well understood by the skilled artisan. In some embodiments, the salts of the inhibitors of the invention are pharmaceutically acceptable salts.

Where tautomeric forms may be present for any of the small molecules described herein, each and every tautomeric form is intended to be included in the present invention, even though only one or some of the tautomeric forms may be explicitly depicted. By way of example, if a 2-hydroxypyridyl moiety is described/depicted, the corresponding 2-pyridone tautomer is also intended.

The invention also includes any or all stereochemical forms, including any enantiomeric or diastereomeric forms of the small molecules described. The recitation of the structure or name herein is intended to embrace all possible stereoisomers of small molecules depicted/described. All forms of the small molecules are also embraced by the invention, such as crystalline or non-crystalline forms of the small molecules. Compositions comprising a small molecule of the invention are also intended, such as a composition of substantially pure inhibitor, including a specific stereochemical form thereof, or a composition comprising mixtures of small molecules of the invention in any ratio, including two or more stereochemical forms, such as in a racemic or non-racemic mixture.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any small molecule of the present invention can be used to promote tendon regeneration.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization, new analogs are designed considering issues of drug delivery, metabolism, novelty, and safety.

In some instances, small molecules described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

In one embodiment, the small molecules described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Alternative Compositions

A skilled artisan should recognize that the compositions of the present invention (i.e., a composition for targeting a tendon regenerating therapeutic to a site in need of tendon regeneration) is not limited to the nanoparticles and/or polymers described herein. The targeting ligands and/or therapeutic agents described herein can be incorporated into any delivery system known in the art suitable for specific and/or targeted delivery of a tendon regenerating therapeutic to a site in need of tendon regeneration (i.e., a damaged, injured, and/or healing tendon).

Representative delivery systems include, but are not limited to, macromers, micelles, hydrogels, microparticles, and liposomes. Examples of these systems and their methods of production are described in U.S. Pat. No. 10,195,284, incorporated by reference herein its entirety.

Representative polymers include, but are not limited to, block copolymers, brush polymers, phosphate-containing polymers, and amino acid mimetic polymers. Examples of these polymers and their methods of production are described in U.S. Pat. No. 10,195,284, incorporated by reference herein its entirety.

### Methods of Preparing Nanoparticles

In some embodiments, the present invention comprises a method of preparing a nanoparticle. In some embodiments, the nanoparticle is useful for targeted delivery of a therapeutic agent to a desired tissue.

In some embodiments, the method comprises forming nanoparticles by self-assembly comprising exchanging the solvent a polymer of the present invention is suspended in with an alternate solvent.

In some embodiments, the method comprises a step of conjugating a polymer of the present invention with a targeting ligand of the present invention prior to forming the nanoparticles. In some embodiments, the method comprises a step of conjugating a polymer of the present invention with a targeting ligand of the present invention after the polymer has been formed into a nanoparticle.

In some embodiments, the targeting ligand is conjugated to the polymer by the formation of an amide bond. In some embodiments, the amide bond is formed between a free nitrogen of the targeting ligand and a carboxylic acid, carboxylic acid halide, or anhydride of the polymer. In some embodiments, the amide bond is formed between a free nitrogen of the polymer and a carboxylic acid, carboxylic acid halide, or anhydride of the targeting ligand.

In some embodiments, the amide bond is formed by reacting the polymer with a coupling reagent and subsequently reacting the activated polymer with the targeting ligand. Examples of peptide coupling reagents include, but are not limited to, diphenyl phosphoryl azide (DPPA), 1-chloro-N,N,2-trimethyl-1-propenylamine, chloro-N,N,N′,N′-bis(tetramethylene)formamidinium tetrafluoroborate, PyClU, Chloro-N,N,N′,N′-tetramethylformamidinium hexafluorophosphate, Fluoro-N,N,N′,N′-tetramethylformamidinium hexafluorophosphate (TFFH), Fluoro-N,N,N′,N′-bis(tetramethyl)formamidinium hexafluorophosphate (BTFFH), phosgene, triphosgene, thiophosgene, N,N′-dicyclohexylcarbodiimide (DCC), N-(3-dimethylaminopropyl)-N′-ethylcarbodiimide (EDC), 1-[3-(dimethylamino)propyl]-3-ethylcarbodiimide methiodide (EDC methiodide), N,N′-diisopropylcarbodiimide (DIC), 1-tert-butyl-3-ethylcarbodiimide (BEC), N-cyclohexyl-N′-(2-morpholinoethyl)carbodiimide metho-p-toluenesulfonate (CMC), N,N′-di-tert-butylcarbodiimide, 1,3-di-p-tolylcarbodiimide, 1,1′-carbonyldiimidazole (CDI), 1,1′-carbonyl-di-(1,2,4-triazole) (CDT), oxalic acid diimidazolide, 2-chloro-1,3-dimethylimidazolidinium chloride (DMC), 2-chloro-1,3-dimethylimidazolidinium tetrafluoroborate (CIB), 2-chloro-1,3-dimethylimidazolidinium hexafluorophosphate (CIP), 2-fluoro-1,3-dimethylimidazolidinium hexafluorophosphate (DFIH), (benzotriazole-1-yloxy)tris(dimethylamino)phosphonium hexafluorophosphate (BOP), (benzotriazole-1-yloxy)-tripyrrolidinophosphonium hexafluorophosphate (PyBOP)®, (7-azabenzotriazole-1-yloxy)tripyrrolidinophosphonium hexafluorophosphate (PyAOP), bromotris(dimethylamino)phosphonium hexafluorophosphate (BroP), chlorotripyrrolidinophosphonium hexafluorophosphate (PyCloP), bromotripyrrolidinophosphonium hexafluorophosphate (PyBroP), 3-(diethoxyphosphoryloxy)-1,2,3,-benzotriazin-4(3H)-one (DEPBT), O-(benzotriazole-1-yl)-N,N,N′,N′-tetramethyluronium hexafluorophosphate (HBTU), O-(benzotriazole-1-yl)-N,N,N′,N′-tetramethyluronium tetrafluoroborate (TBTU), O-(7-azabenzotriazol-1-yl)-N,N,N′,N′-tetramethyluronium hexafluorophosphate (HATU), O-(benzatriazol-1-yl)-N,N,N′,N′-bis(tetramethylene)uronium hexafluorophosphate (HBPyU), O-(benzatriazol-1-yl)-N,N,N′,N′-bis(pentamethylene)uronium hexafluorophosphate (HBPipU), (benzotriazole-1-yloxy)dipiperidinocarbenium tetrafluoroborate (TBPipU), O-(6-chlorobenzotriazol-1-yl)-N,N,N′,N′-tetramethyluronium hexafluorophosphate (HCTU), O-(6-chlorobenzotriazol-1-yl)-N,N,N′,N′-tetramethyluronium tetrafluoroborate (TCTU), O-(3,4-dihydro-4-oxo-1,2,3-benzotriazin-3-yl)-N,N,N′,N′-tetramethyluronium tetrafluoroborate (TDBTU), O-(2-oxo-1(2H)pyridyl)-N,N,N′,N′-tetramethyluronium tetrafluoroborate (TPTU), O-[(etoxycarbonyl)cyanomethylenamino]-N,N,N′,N′-tetramethyluronium hexafluorophosphate (HOTU), O-[(etoxycarbonyl)cyanomethylenamino]-N,N,N′,N′-tetramethyluronium tetrafluoroborate (TOTU), N,N,N′N′-tetramethyl-O-(N-succinimidyl)uronium hexafluorophosphate (HSTU), N,N,N′N′-tetramethyl-O-(N-succinimidyl)uronium tetrafluoroborate (TSTU), dipyrrolidino(N-succinimidyloxy)carbenium hexafluorophosphate (HSPyU), S-(1-oxido-2-pyridyl)-N,N,N′,N′-tetramethyluronium tetrafluoroborate (TOTT), propylphosphonic anhydride, 2-chloro-1-methylpyridinium iodide, 2-chloro-4,6-dimethyoxy-1,3,5-triazine (CDMT), 4-(4,6-dimethoxy-1,3,5-triazin-2-yl)-4-methylmorpholinium chloride (DMTMM), hydroxybenzotriazole (HOBt), and 1-hydroxy-7-azabenzotriazole (HOAt). In some embodiments, a nitrogen of the targeting ligand directly reacts with an anhydride present in the polymer. In some embodiments, a nitrogen of the polymer reacts with an anhydride present in the targeting ligand. In some embodiments, the reaction of the nitrogen and anhydride is an anhydride ring-opening (ARO) reaction.

### Methods

In some embodiments, the present invention comprises a method of administering to a subject in need thereof a composition for use in promoting tendon regeneration. In some embodiments, the invention comprises a method of promoting tendon regeneration at a site of tendon injury in a subject in need thereof. In some embodiments, the invention comprises method of treating tendon injury in a subject in need thereof.

In some embodiments, the subject in need thereof has a disease or disorder associated with tendon injury. Representative embodiments include, but are not limited to, tendonosis, tendonitis, tendinopathy, partial tendon rupture, and complete tendon rupture, age-related tendon degeneration, and comorbidity-related tendon degeneration (e.g., diabetes).

In some embodiments, the method comprises administering to the subject in need thereof a composition for controlled local delivery of a therapeutic agent to injured tendon. In some embodiments, the composition comprises a targeting ligand. In some embodiments, the targeting ligand comprises a peptide that binds to TRAP. In some embodiments, the peptide comprises TBP, as described elsewhere herein. In some embodiments, the targeting ligand is tethered to a polymer. In some embodiments, the polymer comprises poly(styrene-alt-maleic anhydride)-b-poly(styrene) (PSMA-b-PS). In some embodiments, the composition comprises a therapeutic agent that promotes tendon regeneration.

In some embodiments, the composition of the invention is administered prior to, during or after tendon surgery. In some embodiments, the composition of the invention is administered during the late inflammatory and early proliferative stages of a healing tendon injury.

Administration

The compositions of the present invention may be administered in any manner suitable for directing the therapeutic agent to the site in need of tendon regeneration. Modes of administration include, but are not limited to, intravenous, intravascular, intramuscular, subcutaneous, intracerebral, intraperitoneal, soft tissue injection, surgical placement, arthroscopic placement, and percutaneous insertion, e.g., direct injection, cannulation, or catheterization. The methods described herein result in localized administration of a therapeutic agent encapsulated by the nanoparticle to the site or sites in need tendon regeneration. Any administration may be a single application of a composition of invention or multiple applications. Administrations may be to a single site or to more than one site in the individual to be treated. Multiple administrations may occur essentially at the same time or separated in time.

Although the description of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans, other primates, and mammals (including commercially relevant mammals such as non-human primates, cattle, pigs, horses, sheep, cats, and dogs).

It will be appreciated that a composition of the invention may be administered to a subject either alone, or in conjunction with another therapeutic agent.

The pharmaceutical compositions useful for practicing the invention may be administered to deliver a dose of from ng/kg/day and 100 mg/kg/day. In one embodiment, the invention envisions administration of a dose that results in a concentration of the compound of the present invention between 0.1 μM and 10 μM in a mammal.

Typically, dosages which may be administered in a method of the invention to a mammal, (e.g., a human) range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 1 μg to about 10 mg per kilogram of body weight of the mammal. In some embodiments, the dosage will vary from about 3 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

Pharmaceutical Compositions

In some embodiments, the composition of the present invention comprises a pharmaceutical composition. In some embodiments, the therapeutic agent to be delivered to the site in need of tendon regeneration comprises a pharmaceutical composition. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents include growth factors, hormones, anti-inflammatories including corticosteroids, immunosuppressants, and the like.

Controlled- or sustained-release formulations of a pharmaceutical composition of the invention may be made using conventional technology.

For oral application, particularly suitable are tablets, dragees, liquids, drops, or capsules, caplets and gelcaps. Other formulations suitable for oral administration include, but are not limited to, a powdered or granular formulation, an aqueous or oily suspension, an aqueous or oily solution, a paste, a gel, a toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated, or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents, fillers, lubricants, disintegrates, or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups, or suspensions. The liquid preparations may be prepared by conventional means with pharmaceutically acceptable additives such as suspending agents (e.g., sorbitol syrup, methyl cellulose, or hydrogenated edible fats); emulsifying agent (e.g., lecithin or acacia); non-aqueous vehicles (e.g., almond oil, oily esters, or ethyl alcohol); and preservatives (e.g., methyl or propyl p-hydroxy benzoates or sorbic acid). Liquid formulations of a pharmaceutical composition of the invention that are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists of the use of materials that are solid or semi-solid at room temperature (i.e., having a relatively low softening or melting point range) to promote granulation of powdered or other materials, essentially in the absence of added water or other liquid solvents. The low-melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

Tablets may comprise multi-layer tablets comprising a layer providing for the delayed release of one or more compounds of the invention, and a further layer providing for the immediate release of a medication for treatment of a disease. Using a wax/pH-sensitive polymer mix, a gastric insoluble composition may be obtained in which the active ingredient is entrapped, ensuring its delayed release.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parenterally-administrable formulations that are useful include those that comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles that comprise the active ingredient and that have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. By way of example, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Remington's Pharmaceutical Sciences (1985, Genaro, ed., Mack Publishing Co., Easton, PA), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Preparation of TRAP Binding Peptide-Nanoparticles (TBP-NPs)

Developing a drug delivery system with high specificity, optimum drug loading, and outstanding stability that can increase circulation half-life, systemic drug concentrations, and drug bioavailability is critical for controlled drug delivery (Bajo, J., et al., 2021, Current Drug Targets, 22(8):922-946). A targeted drug delivery system has previously been developed by introducing a TRAP binding peptide (TBP) to PSMA-b-PS NPs via carbodiimide conjugation in an aqueous medium. This system effectively delivered a β-catenin antagonist to the fractured femur, however, significant shortcomings exist in the method (Wang, Y., et al., 2017, ACS Nano, 11(9):9445-9458); Chandrasiri, I., et al., 2022, Frontiers in Biomaterials Science, 1:e1003172). The conjugation of the PSMA-b-PS NPs to TBP was carried out assuming that only one functional group from each reactant (amine on TBP) could participate in the reaction, which is typically not the case, as carbodiimide coupling reagents activate numerous reaction sites on multifunctional biomaterials (biomaterials containing multiple amines (TBP)/carboxylate (PSMA-b-PS) functional groups), resulting in an uncontrolled conjugation due to high crosslinking and poor reproducibility (Chandrasiri, I., et al., 2022, Frontiers in Biomaterials Science, 1:e1003172). Thus, an alternative conjugation method that is more controlled, reproducible, and ensures no unintended intramolecular/intermolecular reactions occur is developed. Rigorous characterization methods, to detect involuntary responses to ensure the production of a reliable and reproducible NP drug delivery platform for therapeutic tendon treatment, are utilized.

Poly (Styrene-Alt-Maleic Anhydride)-b-Poly(Styrene) (PSMA-b-PS) Synthesis and Characterization

Amphiphilic PSMA-b-PS polymers were synthesized via reversible addition-fragmentation chain transfer (RAFT) polymerization by dissolving styrene (99%, ACS grade), maleic anhydride, and 4-cyano-4-dodecyl sulfanyltrihiocarbonyl sulfanyl pentanoic acid (DCT) ([styrene]:[maleic anhydride]=4:1) in 1,4 dioxane (128% W/W). RAFT polymerization is a controlled living radical polymerization technique that ensures ideal molecular weights and hydrophobicity of synthesized polymers. 2,2′-Azo-bis(isobutyl nitrite) (AIBN) was recrystallized from methanol and used as an initiator by addition into the reaction cocktail. The reaction was then purged with nitrogen to ensure that there was no formation of an ignitable atmosphere, as well as remove any oxygen that might inhibit the reaction, after which it was placed in a 60° C. oil bath for 72 hours for polymerization. After 72 hours, the reaction was terminated by exposure to air, and the polymer was dissolved in acetone and precipitated in petroleum ether. The precipitated product was dried in a vacuum, after which gel permeation chromatography was used as a characterization method to determine polymer properties (e.g., molecular weight, dispersity).

Peptide Synthesis

A TRAP binding peptide (TBP) with sequence TPLSYLKAllocGLVTVG (SEQ ID NO:3) was synthesized using microwave-assisted solid-phase peptide synthesis. To minimize crosslinking during conjugation, the functional groups were protected with an acid-resistant protecting group that protects the primary lysine amine. A scrambled control peptide (SCP), with the same amino acids as TBP but with a different peptide sequence (VPVGTLSYLLKAllocLTG, SEQ ID NO:4), was also synthesized. The peptides were synthesized on Fmoc-Gly-Wang resin with O-benzotriazole-N,N,N′,N′-tetramethyl-uronium-hexafluorophosphate (HBTU) in DMF and 2 M N,N-diisopropylethylamine (DIEA) in NMP coupling. After synthesis and deprotection, the peptides were cleaved off the resin and precipitated in ice-cold ether to ensure the removal of DMF and byproducts of the cleavage mixture. The resulting peptides were purified by dialysis against water to ensure the removal of trace impurities and truncated peptides. High-performance liquid chromatography (HPLC) and molecular weight, using matrix-assisted laser desorption/ionization-time of flight (MALDI-TOF) mass spectrometry, were used to analyze the purity of synthesized peptides and confirm a molecular weight of 1432 Da, respectively. Only peptides with >95% purity were used for subsequent studies.

Polymer Conjugation Via Anhydride Ring-Opening (ARO) Nucleophilic Addition-Elimination

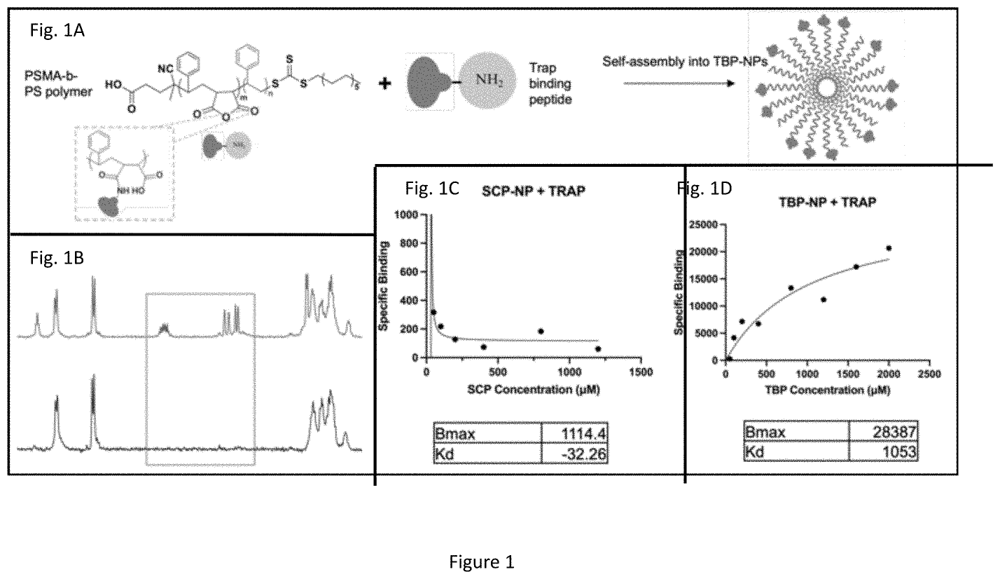

ARO conjugation chemistry was used to conjugate peptides to PSMA-b-PS polymers (FIG. 1A). As the hydrophilic block of PSMA-b-PS contains cyclic anhydrides, the nucleophilicity of primary amines (on the peptide) can promote a nucleophilic addition-elimination reaction. Furthermore, the ARO conjugation technique provides a facile yet robust approach without activators and eliminates additional purification steps. Based on the number of carboxylate groups on the polymer chain, an anhydride feed ratio of 10% was used for TBP and SCP conjugation. The conjugation reaction was carried out in a non-nucleophilic organic solvent, DMF, under nitrogen. After the conjugation of peptides to the polymer, deprotection of the primary amine was then performed to allow binding to TRAP. Nuclear Magnetic Resonance spectroscopy was used to analyze deprotected peptide-polymer conjugates to ensure the absence of allylic groups present from the Alloc protecting group (FIG. 1B).

Characterization of PSMA-b-PS Conjugates

Gel permeation chromatography (GPC) was used to evaluate the number-average molecular weight (Mₙ), weight-average molecular weight (M_{w}), and dispersity (Ð) of the conjugated polymers (PSMA-b-PS-TBP, PSMA-b-PS-SCP). Crosslinking was quantitatively deduced by comparing the GPC results of the polymer to polymer-peptide conjugates. The molar mass differences between the PSMA-b-PS polymer and PSMA-b-PS-TBP/PSMA-b-PS-SCP were used to calculate the number of conjugated peptides. Peptide-polymer conjugates with PDI>1.2 and “conjugation efficiency”>150%, indicating high incidence of crosslinking, were not used for subsequent studies.

Effect of Feed Ratio on TBP-NP Formation

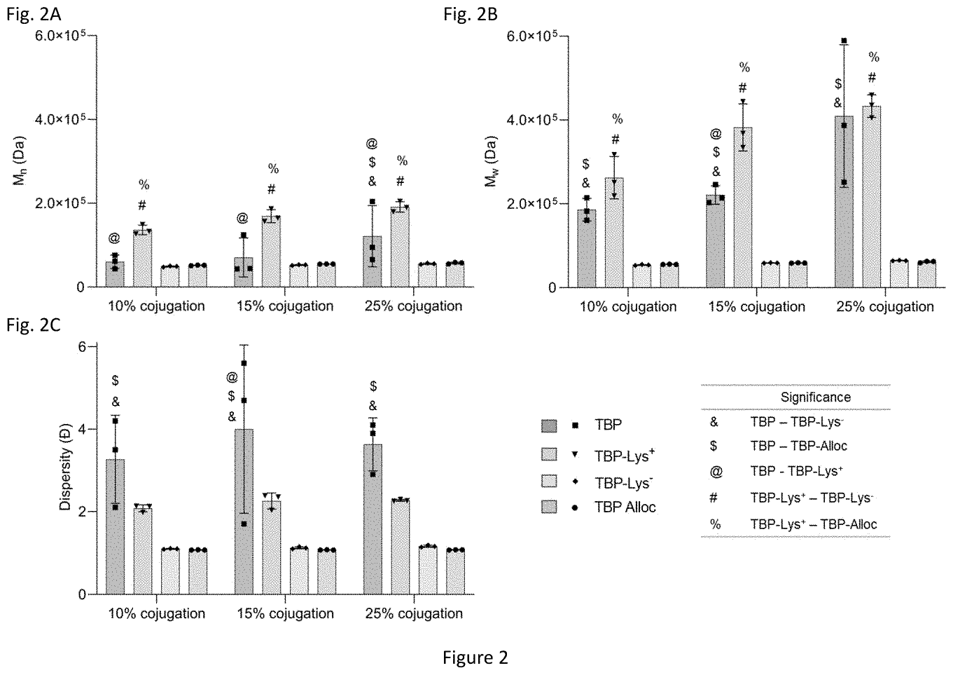

To determine the optimum conditions for ARO-based formation of TBP-NPs, ARO conjugation of TBP to polymer was conducted under various conditions. Additionally, several varieties of TBP were examined, including normal TBP (TBP), Alloc-protected TBP (TBP Alloc), TBP without lysine (TBP-Lys⁻), and TBP with an extra lysine (TBP-Lys⁺). TBP-polymer conjugates were prepared with TBP at 10%, 15%, or 25% of the polymer with each TBP derivative. While TBP and TBP-Lys⁺ demonstrated increasing Mₙ and M_{w} with increasing feed ratio, TBP-Lys⁻ and TBP Alloc demonstrated no significant change (FIGS. 2A and 2B). Additionally, while the dispersity was higher for TBP-Lys⁺ than TBP-Lys⁻ and TBP Alloc, TBP yielded the highest dispersity (FIG. 2C).

Self-Assembly of NPs and Characterization:

Polymer-peptide conjugates were self-assembled into nanoparticles via solvent exchange from dimethylformamide (DMF) to water using a syringe pump. Dynamic light scattering (DLS) was used to characterize the size and zeta potential of TBP-NPs and SCP-NPs. An in vitro binding assay that involved exposure of TBP-NPs and SCP-NPs to TRAP in serial dilutions was used to determine the binding affinity of TBP-NPs and establish their efficacy for in vivo applications. Specifically, after exposure of TBP-NPs to TRAP and incubation for 2 hours, fluorescamine, a spiro compound that reacts with primary amines to form highly fluorescent compounds, was added to the reaction. The protein-dye complex was excited with UV LED (365 nm) and emitted at 470 nm on a Cytation 5 Imaging Multimode Reader. The binding affinity (Kd) and maximum binding (Bmax) were then calculated to determine the efficacy of the synthesized TBP-NPs (FIG. 1 C-D).

Preparation of TBP-NPs by Tagging of Pre-Prepared NPs

As a comparison, nanoparticles were also prepared by a comparison, alternate methods of preparing TBP-NPs were investigated. In one method, the NPs were prepared prior to tagging with TBP for targeting. In this method, PSMA-b-PS was prepared through RAFT polymerization as previously described. Here, however, the nanoparticles were formed directly from the free polymers by solvent exchange. The nanoparticles were then tagged with TBP via traditional peptide-coupling, via carbodiimide chemistry, yielding the expected amide bonds. With this approach, however, it is possible for TBP to bond with a nanoparticle through either the N-terminal primary amine or through the lysine primary amine. As such, the conjugation efficiencies of both free TBP and the Alloc-protected-lysine TBP were compared. The Alloc-protected-lysine TBP group yielded higher conjugation efficiency with minimal crosslinking compared to free TBP, where there was markedly higher crosslinking.

In a second method, TBP-NPs were prepared by conjugation of TBP to polymer prior to formation of nanoparticles, however in this instance unprotected TBP was utilized.

The TBP-NPs produced by these methods were then analyzed to characterize the properties of both the TBP-polymer conjugates produced as well as the overall nanoparticle (Table 1).

TABLE 1
Properties of NPs and polymers.

| | | ARO-TBP- | | CDI-TBP- | Expected |
|---|---|---|---|---|---|
| Conjugation Type | CDI-TBP | Alloc | ARO-TBP | Alloc | Value |
| Number-Average | 293,000 | 51,910 | 60,440 | 45,000 | 54,820 |
| Polymer Molecular | | | | | |
| Weight (Da) | | | | | |
| Dispersity | 5.2 ± 0.8 | 1.1 ± 0.1 | 3.3 | 1.2 ± 0.1 | <1.2 |
| Diameter (nm) | 66 ± 3 | 32 ± 2 | N/A | 33 ± 3 | ~30 |
| Dispersity for Size | 0.4 ± 0.1 | 0.1 ± 0.1 | N/A | 0.2 ± 0.1 | <0.3 |
| Binding Affinity | Not | 186 μM | N/A | 468 μM | Sub-nM |
| (Kd) | Detected | | | | range |

N/A: experimental results not yet available.

### Example 2: Controlled Localized Delivery of Drugs for Tendon Regeneration

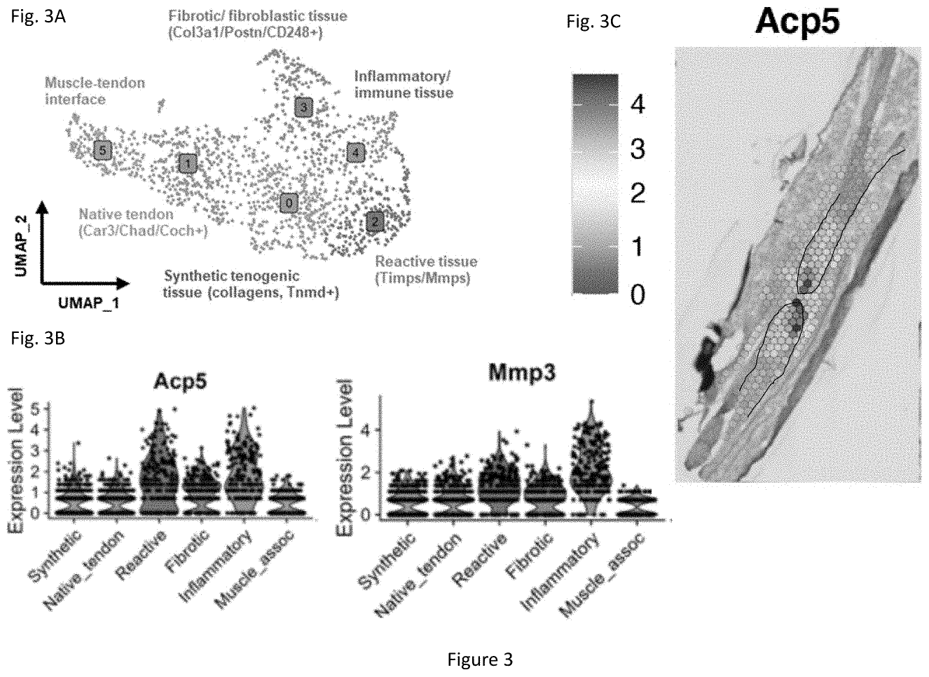

In previous work, technology was developed that showed that introducing TRAP (tartrate resistant acid phosphatase) Binding Peptide (TBP) to a poly(styrene-alt-maleic anhydride)-b-poly(styrene) (PSMA-PS) nanoparticle drug delivery system results in high affinity targeting of TRAP+ cells in vitro and in vivo. These TBP nanoparticles (TBP-NPs) have been leveraged to target areas of high TRAP activity including the fracture healing callus and actively resorbing bone during leukemic progression. While TRAP is typically associated with osteoclasts, other myeloid cells can also express TRAP. Indeed, using spatial transcriptomic profiling of the tendon healing process (FIG. 3), a spatio-molecular cluster associated with inflammatory tissue at the injury site was identified, and this cluster was defined by expression of Acp5, the gene encoding TRAP. Moreover, the present data demonstrate robust TRAP activity in the healing tendon during the late inflammatory and early proliferative stages of healing and that TBP-NP can accumulate at tendons during those timeframes. Thus, while not being bound by scientific theory, it is believed that drug delivery via TBP-NPs may enhance healing of injured tendons.

Acp5 Defines an Inflammatory Tissue Cluster at the Tendon Repair Site:

The fundamental cellular and molecular mechanisms that drive scar-mediated tendon are not well defined. Therefore, the 10× Spatial Transcriptomics platform was utilized to comprehensively define how the spatio-molecular program shifts during healing. Using integrated analysis of data from uninjured, day 14 post-repair, and day 28 post-repair mice, 5 distinct spatio-molecular clusters were identified in the tendon (FIG. 3A). In addition to providing greater resolution of the spatially-dependent fate trajectory of tenocytes, an inflammatory cluster located at the interface between reactive remodeling tendon and the bridging scar tissue was also identified (Cluster 4, FIG. 3A). This cluster is enriched for macrophage markers including Mmp9 and Mmp13. However, the most differentially expressed gene in this cluster is Acp5, the gene encoding TRAP (FIG. 32B). Spatial mapping of Acp5 expression at day 14 demonstrates robust Acp5 expression in both tendon stubs and the bridging scar tissue (FIG. 3C).

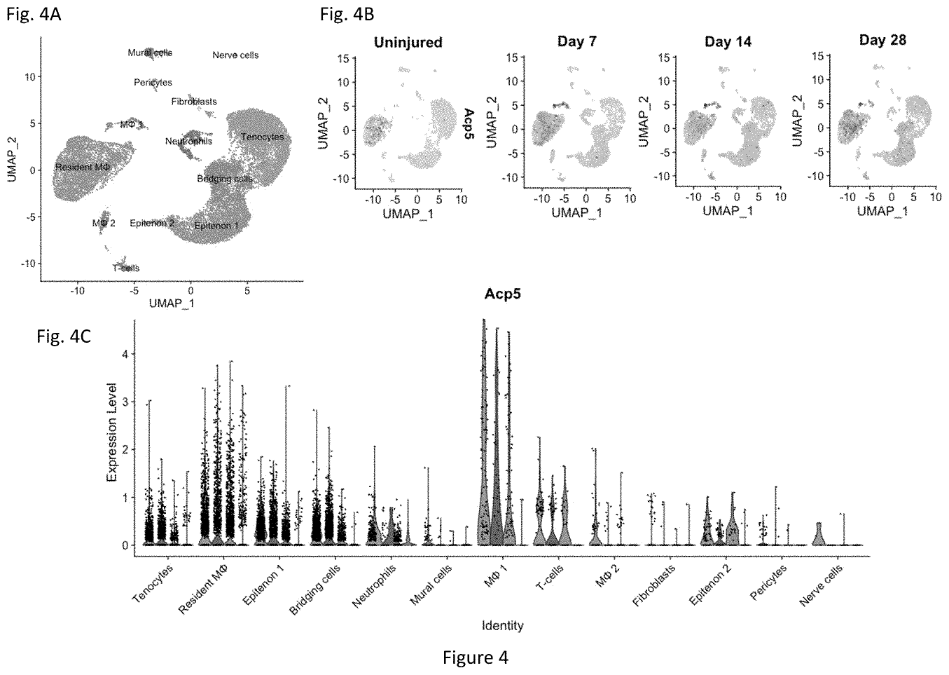

This was further validated in additional single-cell data (FIG. 4), which demonstrated that macrophage 1 cells undergo a dramatic increase in the expression of Acp5, which is not mimicked in other cell types (FIG. 4B). Of note is the observation that macrophage 2 cells, unlike macrophage 1 cells, do not undergo this robust increase in Acp5 expression (FIG. 4C), highlighting the potential specificity of targeting.

Reduction in S100a4 Promotes Enhanced Healing

In the liver, macrophage derived S100a4 cells have been shown to prompt the conversion of resident cells into alpha SMA positive myofibroblasts, resulting in increased fibrosis (Chen, L., et al., 2015, Journal of Hepatology, 62(1):156-164). In the tendon, knockdown of S100a4 has resulted in reduced fibrosis (Ackerman, J. E., et al., 2019, eLife, 8:e45342).

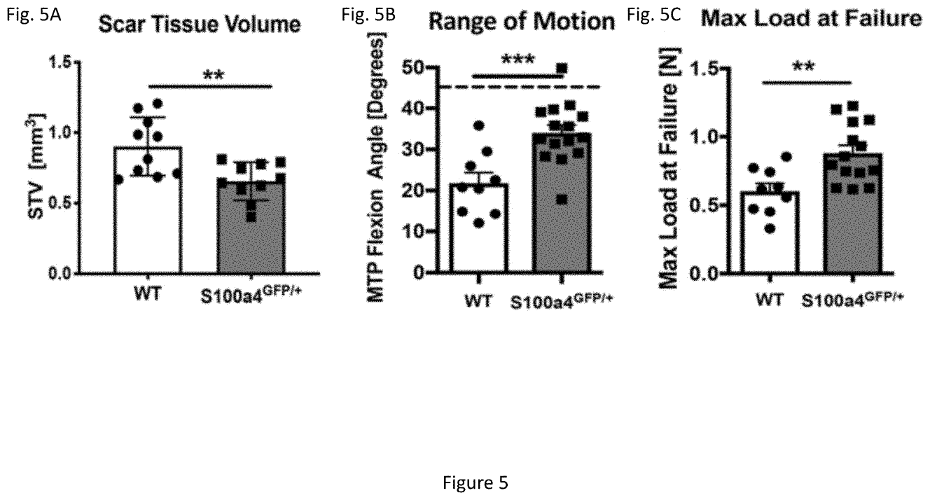

Wildtype and S100a4^{GFP/+} C57Bl/6J mice underwent flexor tendon transection and repair surgery underwent tendon repair. The mice were examined for healing 14 days post-surgery. In testing for physiological responses, S100a4^{GFP/+} the tendon had a significantly improved range of motion and were capable of bearing more weight (FIGS. 4B and 4C). The injured tendon of S100a4^{GFP/+} mice further demonstrated a significant decrease in scar tissue volume (FIG. 5A). Combined, these demonstrate that knockdown of S100a4 promotes tendon healing.

Abundant TRAP+ Cells are Present During the Late Inflammatory and Early Proliferative Phases of Tendon Healing:

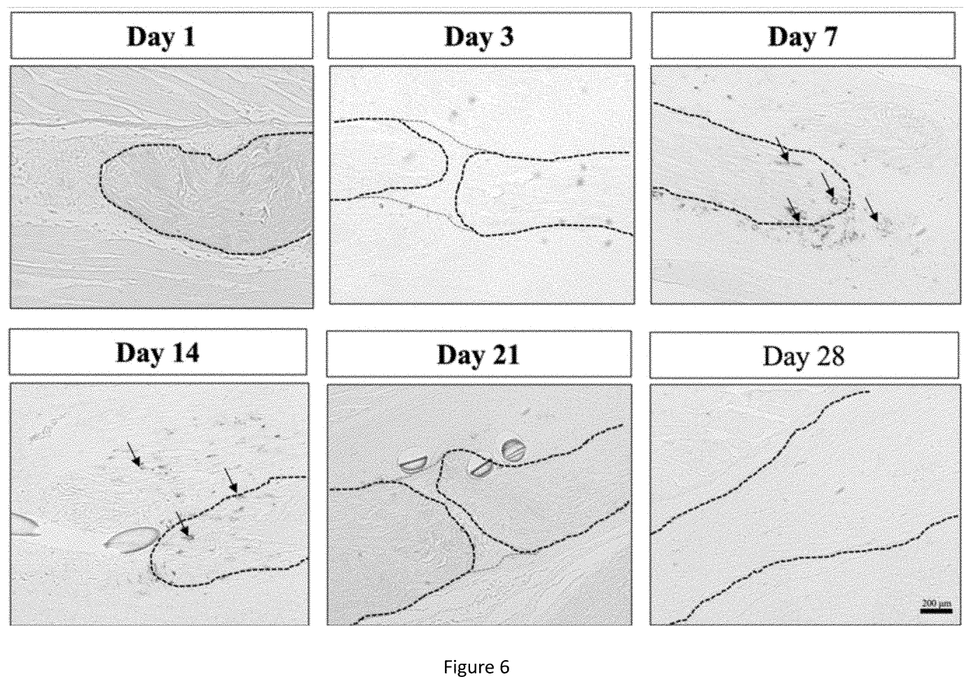

Based on the surprising finding of high Acp5 expression in the healing tendon, TRAP staining was conducted to identify areas of TRAP activity. No TRAP activity was observed in uninjured tendon, and minimal TRAP activity was observed prior to day 7 post-surgery. By day 7 several TRAP+ cells were observed in the bridging tissue between the tendon stubs (FIG. 6). At day 14 a substantial increase in TRAP staining was observed, with TRAP activity observed diffusely throughout the native tendon stub (FIG. 6), and in the bridging tissue between the tendon stubs (FIG. 6). Collectively, these data demonstrate robust TRAP activity in the healing tendon, supporting the immense translational potential of using TBP-NPs for high efficiency drug delivery to the healing tendon.

TRAP-Binding-Peptide Laden Nanoparticles Enhance Homing to the Healing Tendon:

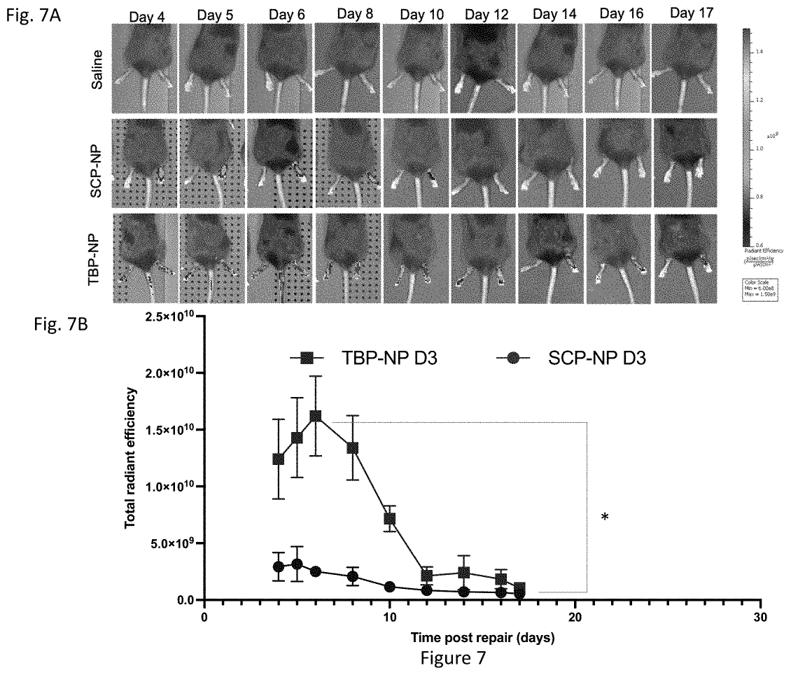

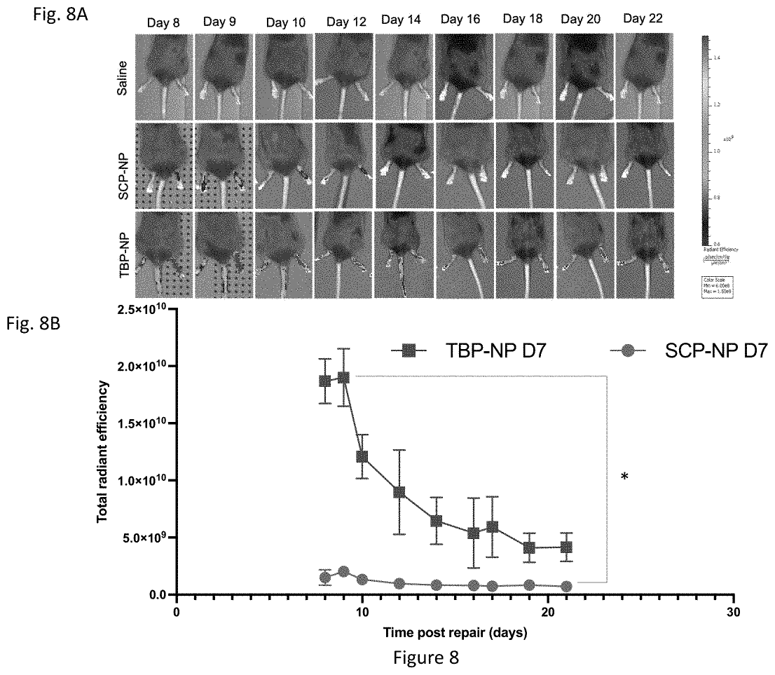

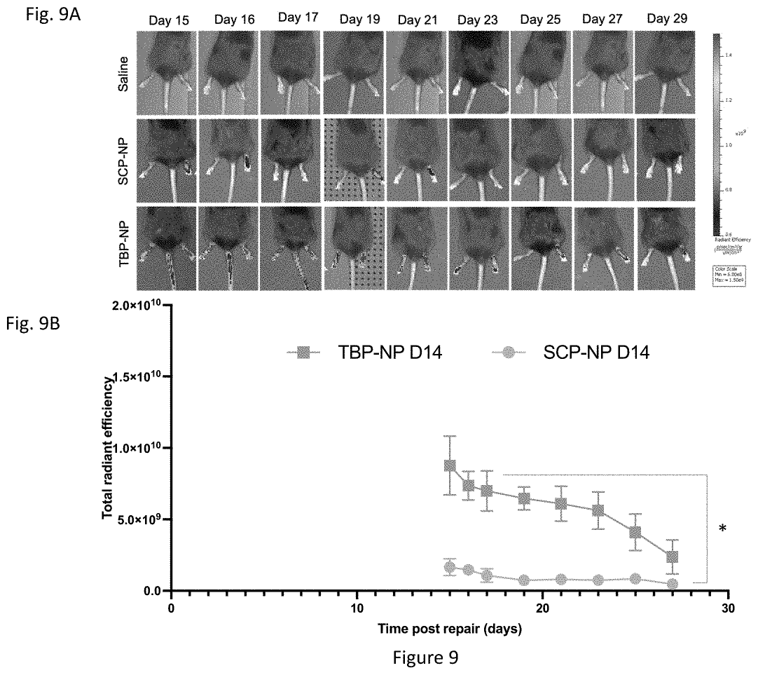

To demonstrate the feasibility of TBP-NPs enhancing NP localization at the healing tendon, C57Bl/6J mice underwent flexor tendon transection and repair surgery. On days 3, 7, and 14 post-tendon repair surgery, IR780-labelled TBP-NPs or Scrambled peptide nanoparticles (SCP-NPs) were administered via retroorbital injection. Saline injection was used as a negative control. An in vivo imaging system (IVIS) was used to determine the extent of localization to the tendon repair site between 1 and 14 days after NP injection. While both TBP-NPs and SCP-NPs homed to the tendon repair site, TBP-NPs resulted insignificantly increased accumulation, as well as prolonged retention, relative to SCP-NPs (FIGS. 6-9). The highest accumulation of TBP-NPs was observed in the day 7-treated mice (˜four-fold over SCP-NP), resulting in sustained TBP-NP retention for 14 days (FIG. 8). In contrast, treatment at day 3 resulted in high accumulation (˜three-fold vs SCP-NP) but a drastic reduction in signal at day 8, indicating poor retention when delivered on day 3 (FIG. 7). Finally, delivery at day 14 decreased initial accumulation relative to days 3 and 7 and resulted in relatively rapid clearance HO days, FIG. 9).

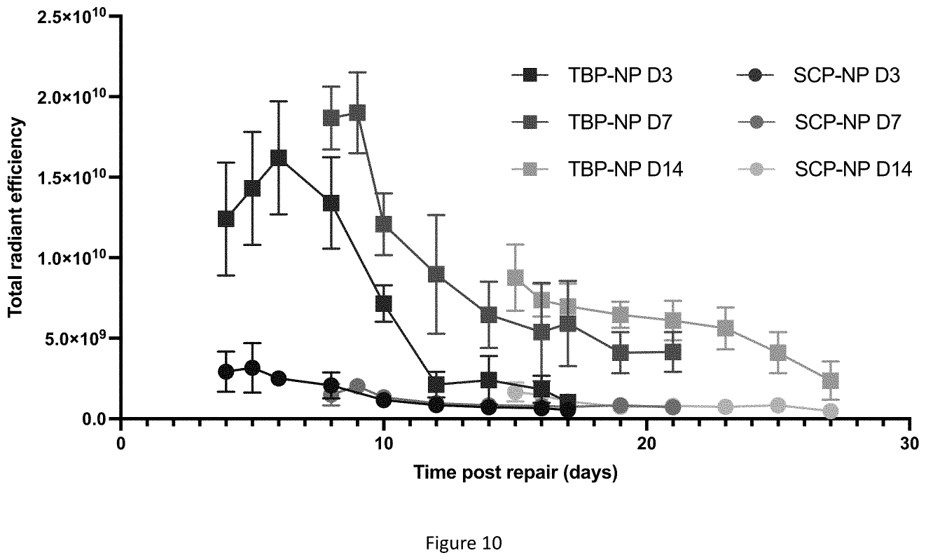

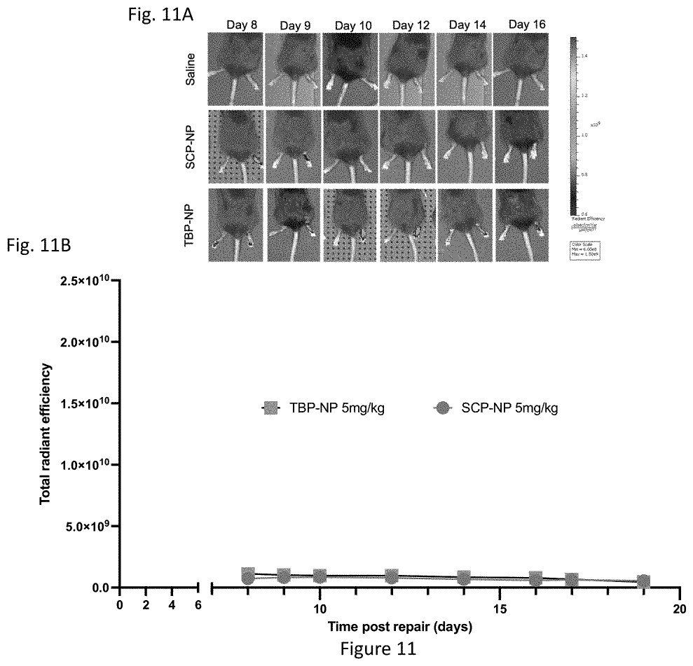

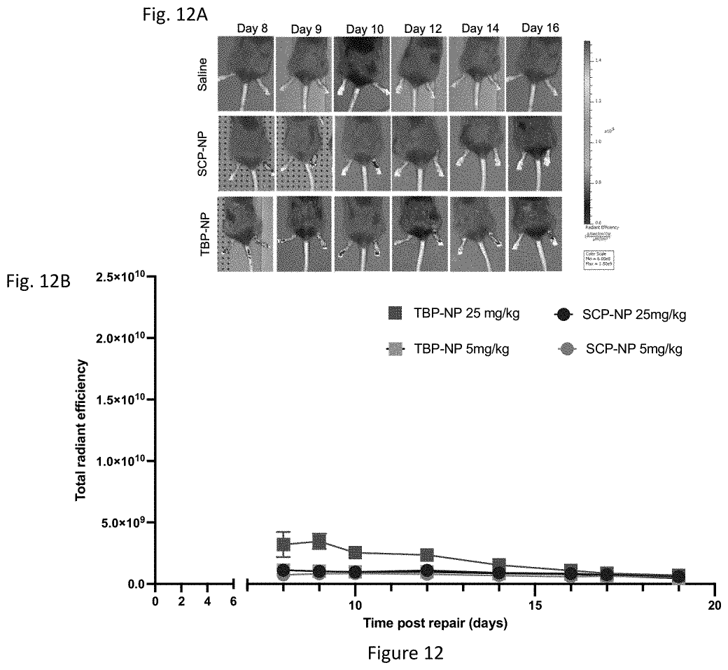

To investigate the optimum dose that ensures high tendon targeting with minimal off-target accumulation, three NP dose concentrations were investigated; Mice received 5, 25, or 50 mg/kg doses of TBPIR780-NP and SCP-IR780-NPs to study dose dependent accumulation and cellular uptake at the tendon repair site. Live animal imaging via XENOGEN/IVIS imaging system was used to longitudinally analyze NP biodistribution in the tendon from 24 hours until signal loss after NP administration (FIGS. 10-12). To investigate in vivo distribution more closely, tendons and other tissues (liver, spleen, lungs) are harvested and imaged with IVIS, frozen-sectioned, and counterstained with DAPI. Confocal imaging determines the extent of tendon targeting, NP retention, and off-target accumulation 24 hours post-surgery. Furthermore, a cytotoxicity assay involving detection of liver enzymes namely alanine transaminase (ALT), aspartate aminotransferase (AST), as well as kidney enzymes namely creatinine and urate in blood serum levels are used to confirm optimum dosing conditions.

Cellular Uptake

To investigate in vivo NP uptake at a cellular level, Scx-Cre; Ai9 mice are injected once with fluorescently labeled NPs (TBP-IR780-NP and SCP-IR780-NP) (50 mg/kg) and saline after repair surgery, followed by cell isolation (24 hours after treatment) from repaired tendons and contralateral controls for flow cytometry analysis. Cells are isolated by enzymatic digest and incubated with specific cell markers, including CD45+/F4/80+/Gr-1− for macrophages, CD45+/F4/80−/Gr-1+ for neutrophils, and CD14/CD16 for monocytes, per established protocols. Tenocytes are identified by positive expression of tdTomato, which marks cells that express Scx (Ackerman, J. E., et al., 2021, bioRxiv, Art. 446663). The influence of NP timepoint administration, dosage, and frequency on cellular uptake is investigated. The integrated fluorescent signal from individual cells is measured by side scattering and interpreted as either an “NP containing cell” or an “NP free cell.” Pictures of each cell, obtained via ImageStream flow cytometry, are used to confirm NP location and intensity. Representative flow cytometry histograms and images of these cell types at different treatment times are used to quantify uptake. Histology quantifies cellular uptake and NP spatial localization by staining for macrophages (F4/80) and tenocytes (TdTomato).

In addition to demonstrating the ability to efficiently deliver TBP-NPs to the tendon repair site, these data also suggest minimal recruitment of TBP-NPs to bone despite some persistent TRAP activity during normal bone remodeling. However, a research strategy has been proposed that will rigorously track bone targeting of TBP-NPs and the potential off-target effects of TBP-NP delivery of promising tendon regenerative drugs, including but not limited to, Niclosamide.

### Example 3: TBP-NP-Mediated Delivery of Niclosamide

Niclosamide is an FDA-approved anthelminthic effective against tapeworms (Chen, W., et al., 2018, 41:89-96). The ability to inhibit the S100a4 gene and protein expression was determined in a high throughput screen of the Library of Pharmacologically Active Compounds 1280 using an S100a4-luciferase construct (Sack, U., et al., 2011, JNCI, 103(13):1018-1036).

TBP-NP-Niclosamide Loading Efficiency and Capacity

Niclosamide is solubilized in chloroform and loaded into NPs. Loading efficiency and capacity are characterized as previously described. Specifically, different combinations of drug amount, carrier volume and power, and NP amount are tested for maximum loading efficiency. Niclosamide loading efficiency and capacity are quantified using HPLC.

Release of Niclosamide from TBP-NPs

Drug-loaded NPs are dialyzed at neutral pH in 1×PBS (pH 7.4) and acidic pH (pH 4.5) using MWCO 6-8 kDa dialysis membranes to emulate pH changes that occur during endolysosomal trafficking during intracellular delivery. Release buffer is changed twice daily, and 200 μL of NP-drug solution is collected over nine days. The drug release is be quantified using HPLC.

In Vitro S100a4 Inhibition and Functional Assay

An ELISA assay is used to confirm inhibition of S100a4 in both bone marrow derived macrophages and tenocytes isolated from S100a4^{GFPpromoter} mice. Bone marrow derived macrophages (BMM) have been shown to exhibit chemotactic migration due to the presence of macrophage-colony stimulating factor (CSF-1), hence, to functionally confirm s100a4 inhibition, BMMs treated with drug loaded nanoparticles are cultured in a transwell assay, and the number of cells that migrate to the bottom of the transwell is counted. As a control, BMMs treated with TBP-NP-Niclosamide are also cultured in a transwell in the presence of CSF-1, and migration is assessed.

In-Vivo TBP-NP-Niclosamide-Induced Healing

S100a4^{GFPpromoter} mice (#12893, Jackson laboratories) express GFP under the control of endogenous S100a4 promoter such that GFP can be used as a readout of active S100a4 expression. S100a4^{GFP} mice undergo flexor tendon injury and repair surgery at 10-12 weeks. Mice are treated with TBP-NP-niclosamide via retro-orbital injection on day 7 post-surgery. Saline and the free drug are also administered to evaluate the impact of injected drug/NP on tendon healing. Significantly, this treatment regimen is based on high TBP-NP accumulation and retention (FIG. 9), S100a4 expression, and S100a4^{GFP+} cells during healing. Healing tendons are harvested from TBP-NP-Niclosamide treated experimental mice and TBP-NP-treated control mice between 14-63 days post-surgery.

Liver and kidney enzymes, namely, ALT and AST, and creatinine and urate are assessed using an Elisa assay. The liver, kidney, spleen, lungs, and heart are also harvested to investigate tissue morphology. To investigate off-target effects in the bone due to TRAP+ osteoclasts, the femur is also harvested for μCT to determine bone volume after drug treatment. Quantification of bone mineral density and bone volume is performed to ensure that there are no off-target effects.

Following harvest, the FDL tendon is isolated at the myotendinous junction. The proximal tendon is secured in tape using cyanoacrylate, and a range of weights (0-19 g) are applied to induce flexion of the digits. The metatarsophalangeal (MTP) joint angle is measured to derive two parameters of scar formation: MTP flexion angle, the degree of flexion at 19 g, and Gliding Resistance, a measure of the ROM over the applied loads. A lower MTP flexion angle and higher Gliding Resistance indicate increased scar tissue formation and impaired gliding function.

Changes in mechanical properties are assessed via endpoint measurements from 14-63 days post-surgery. Structural and material property changes are determined via tensile testing following isolation of the healing FDL after ROM testing. The FDL is tested in tension until failure, and force displacement and stress-strain data are plotted and analyzed for structural and material properties. Photographs acquired from orthogonal orientations are used to determine specimen gauge length and cross-sectional area.

Healing tendons are harvested for paraffin histology and co-immunofluorescent (Co-IF) studies over 14-63 days post-surgery from TBP-NP-niclosamide- and TBP-NP-treated S100a4GFP mice. Serial five-micron paraffin tissue sections are cut through the sagittal plane of the healing tendon in the intact hind paw. To assess tissue morphology, adjacent sections are stained with Alcian Blue/Hematoxylin, Orange G (ABHOG), and Masson's Trichrome. Edu labeling occurs 4 hours prior to harvest with imaging after click-it Edu staining to evaluate proliferation. Changes in apoptosis are assessed via staining for TUNEL and Cleaved Caspase 3. Overall changes in the cellular environment consistent with more regenerative tendon healing, such as accelerated clearance of macrophages and myofibroblasts and enhanced tenogenesis, are assessed via immunofluorescence for pan-macrophage markers (CD68, F4/80), markers of M1 (iNOS, TNFα, CD86, CD64) and M2 (CD206, Arg1, IL1ra, CD163) macrophage polarization, mature myofibroblasts (αSMA), and tenogenesis (Scx, Mkx, Tnmd).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for controlled local delivery of a therapeutic agent to injured tendon, the composition comprising a targeting ligand tethered to a polymer and a therapeutic agent, wherein the therapeutic agent promotes tendon regeneration.

2. The composition of claim 1, wherein the targeting ligand comprises a targeting ligand that specifically binds to a target associated with a site in need of tendon regeneration.

3. The composition of claim 2, wherein the targeting ligand is selected from the group consisting of: a nucleic acid, a peptide, an antibody, an antibody fragment, an inorganic molecule, an organic molecule, and any combination thereof.

4. The composition of claim 3, wherein the targeting ligand comprises a peptide that specifically binds to tartrate-resistant acid phosphatase (TRAP).

5. The composition of claim 4, wherein the targeting ligand comprises TRAP Binding Peptide (TBP).

6. The composition of claim 5, wherein the targeting ligand comprises an amino acid sequence at least 95% identical to SEQ ID NO: 1.

7. The composition of claim 6, wherein the targeting ligand comprises the amino acid sequence of SEQ ID NO: 1.

8. The composition of claim 1, wherein the therapeutic agent comprises one or more selected from the group consisting of: a nucleic acid, a peptide, an antibody, an antibody fragment, an inorganic molecule, an organic molecule, and any combination thereof.

9. The composition of claim 8, wherein the therapeutic agent comprises one or more selected from the group consisting of: a RAGE inhibitor, a RAGE receptor antagonist, an S100A4 inhibitor, a NFκB inhibitor, a NFκB-p65 inhibitor, a ROCK inhibitor, a TGF-β1 receptor antagonist, and an agent that reduces SMAD expression.

10. The composition of claim 9, wherein the therapeutic agent comprises one or more selected from the group consisting of: azeliragon, FPS-ZMI, niclosamide, pentamidine, Daxx, helenalin, parthenolide/micheliolide, Y27632, suramin, and halofuginone.

11. The composition of claim 1, wherein the polymer is selected from the group consisting of poly(ethylene glycol) (PEG) methacrylate and poly(styrene-alt-maleic anhydride)-b-poly(styrene) (PSMA-b-PS).

12. A method of administering to a subject in need thereof a composition for use in promoting tendon regeneration, the method comprising administering to the subject the composition of claim 1.

13. A method of promoting tendon regeneration at a site of tendon injury in a subject in need thereof, the method comprising administering to the subject a composition for controlled local delivery of a therapeutic agent to injured tendon, the composition comprising a targeting ligand tethered to a polymer and a therapeutic agent, wherein the therapeutic agent promotes tendon regeneration.

14. The method of claim 13, wherein the subject has a disease or disorder selected from the group consisting of: tendonosis, tendonitis, tendinopathy, partial tendon rupture, and complete tendon rupture, age-related tendon degeneration, and comorbidity-related tendon degeneration.

15. The method of claim 13, wherein the composition is administered during the late inflammatory and/or early proliferative stages of healing.

16. A method of treating tendon injury in a subject in need thereof, the method comprising administering to the subject a composition for controlled local delivery of a therapeutic agent to injured tendon, the composition comprising a targeting ligand tethered to a polymer and a therapeutic agent, wherein the therapeutic agent promotes tendon regeneration.

17. The method of claim 16, wherein the subject has a disease or disorder selected from the group consisting of: tendonosis, tendonitis, tendinopathy, partial tendon rupture, and complete tendon rupture, age-related degeneration, and comorbidity-related degeneration.

18. The method of claim 16, wherein the composition is administered during the late inflammatory and/or early proliferative stages of healing.


SEQUENCE LISTING (4 sequences)

SEQ ID NO: 1 - protein, 13 aa, synthetic construct
TPLSYLKGLVTVG

SEQ ID NO: 2 - protein, 12 aa, synthetic construct
TPLSYLKGLVTV

SEQ ID NO: 3 - protein, 13 aa, synthetic construct
  MOD_RES 7: note = Alloc Protected
TPLSYLKGLVTVG

SEQ ID NO: 4 - protein, 14 aa, synthetic construct
  METAL 11: note = Alloc Protected
VPVGTLSYLLKLTG